(19) United States
(12) Patent Application Publication
(10) Pub. No.: US 2023/0149547 A1
(43) Pub. Date: May 18, 2023

(54) REDUCED CALORIC INTAKE AND IMMUNOTHERAPY FOR THE TREATMENT OF CANCER

(71) Applicant: IFOM FONDAZIONE ISTITUTO FIRC DI ONCOLOGIA MOLECOLARE, Milano (MI) (IT)

(72) Inventors: Valter LONGO, Milano (MI) (IT); Salvatore CORTELLINO, Milano (MI) (IT)

(73) Assignee: IFOM FONDAZIONE ISTITUTO FIRC DI ONCOLOGIA MOLECOLARE, Milano (MI) (IT)

(21) Appl. No.: 17/916,068

(22) PCT Filed: Apr. 1, 2021
(86) PCT No.: PCT/EP2021/058656

§ 371 Date: Sep. 30, 2022

(30) Foreign Application Priority Data
Apr. 3, 2020 (IT) 102020000007153

(51) Int. Cl. A61K 45/06; C07K 16/28; A61P 35/00

(52) U.S. Cl. CPC A61K 45/06; C07K 16/2818; C07K 16/2827; C07K 16/2878; A61P 35/00; A61K 2039/507

(57) ABSTRACT

The present invention relates to at least one reduced caloric intake cycle and at least one immunotherapeutic agent for use in the treatment of cancer. Preferably the cancer is characterized by resistance or partial response to the treatment with at least one immunotherapeutic agent.

### FIELD OF THE INVENTION

The present invention relates to at least one reduced caloric intake cycle and at least one immunotherapeutic agent for use in the treatment of cancer. In particular, the agent is a PD-1 inhibitor, a PDL-1 inhibitor, a CTLA4 inhibitor or an OX40 activator.

### BACKGROUND OF THE INVENTION

Tumours develop different strategies to evade the immune response in order to promote growth and metastasis. Tumour hijacks immune checkpoint pathways, developed to limit inflammatory and immune responses, by expressing programmed death-ligand 1 (PD-L1) on their cells surface, and by reshaping the tumour immune microenvironment in order to protect itself from the immune cell mediated T cell cytotoxicity.

Tumour immunogenicity varies between cancer, however high level of tumour infiltrating lymphocytes (TILs) is associated with a better prognosis in cancer patient (Foley E J, 1953; Smith H J, 1966). In cancer the immune checkpoint activation and the immunosuppressive microenvironment render TILs dysfunctional and exhausted. TILs express multiple co-inhibitory or checkpoint receptors, such as cytotoxic T lymphocyte antigen-4 (CTLA-4), programmed cell death-1 (PD-1), T cell immunoglobulin and mucin-domain containing-3 (TIM-3), and lymphocyte-activation gene (LAG-3) (Walunas T L et al., 1994; Fourcade J et al., 2010; Matsuzaki J et al., 2010). The blockade of these checkpoint reverse TILs exhaustion and improve cytotoxicity and proliferation of these cells.

Immune checkpoint inhibitors (ICI) therapy, including antibodies against PD-1, PD-L1 and CTLA-4, achieve substantial and durable response across many tumour types. However only a subset of cancer patients responds to ICI therapies. For instance, the highest response rates to single-agent PD-1 blockade therapy occur in Hodgkin's lymphoma with a range between 80 to 90%, whereas in melanoma patients the response rate is between 35 to 40%. In NSCLC and head and neck, gastroesophageal, bladder and urothelial cancers, hepatocellular carcinoma and renal cell carcinoma the response rates ranges from 15 to 25% (Topalian S L et al., 2012; Garon E B et al., 2015; Motzer R J et al., 2015; Rosenberg J E et al., 2016; Wolchok J D et al., 2017; El-Khoueiry A B et al., 2017; Kim S T et al., 2017; Ribas A & Wolchok J D, 2018).

CTLA-4 monotherapy produces durable responses in 22% of melanoma patients (Schadendorf D et al., 2015), whereas the response rate is below 10% in other solid tumors including pancreatic adenocarcinoma, renal cell carcinoma (Yang J C, et al., 2007), B-cell lymphoma (Lesokhin A M Lesokhin A M et al., 2016), prostate cancer (Slovin S F et al., 2013), refractory colorectal cancer, hepatocellular carcinoma (Sangro B et al., 2013), and malignant mesothelioma (Calabro L et al., 2013).

The anti-CTLA-4 and anti-PD-1 combined treatment achieve high response rate of 58% in melanoma patients (but was associated with severe toxicity (Larkin J et al., 2015; Postow M A, 1015). Ongoing clinical trials are testing the efficacy of PD-1 and CTLA-4 in patients affected by renal cell carcinoma and MSI-high colorectal cancer (Sade-Feldman M. et al., 2017).

The most common adverse event encountered for PD-1/PD-L1 and CTLA-4 pathway inhibitor are fatigue, diarrhoea, rash, and pruritus in 15 to 20% of patients (Robert C et al., 2015; Garon E B et al., 2015; Ribas A et al., 2016; Rosenberg J E et al., 2016; Robert C et al, 2015), whereas smaller percentage of patients develop endocrinopathies, such as thyroid disorders (10 to 15%), hypophysitis, adrenal gland disorders (1 to 3%), and type 1 diabetes (1%), or visceral organ inflammatory toxicities (˜1%) including encephalopathy, meningitis, pneumonitis, myocarditis, esophagitis, colitis, hepatitis, and nephritis, in addition to myositis and arthritis (Sarnaik, A. A. et al, 2011; Wolchok, J. D. et al. 2010).

Anti-PD-1 anti CTLA-4 combined treatment show grade 3-4 adverse event in 59% of melanoma patients, with higher incidence of gastrointestinal event (Wolchok J D, 2017).

Failure of ICI therapy can result from inadequate T-cell priming. Full T-cell activation depends on the interaction of T-cell receptor (TCR) with peptide bound to major histocompatibility complex, but also on costimulatory signals provided by non-T accessory cells (Mueller D L et al, 1989; Esensten J H, 2016). Costimulatory receptors belong to either the immunoglobulin superfamily (e.g., CD28, ICOS, and CD226) or the tumour necrosis factor receptor superfamily (TNFRSF), including CD27, OX40 (also referred to as CD-134), 4-1BB, glucocorticoid-induced TNF receptor related protein, death receptor 3, and CD30 (Dougall W C et al., 2017; Wikenheiser D J et al., 2016; Ward-Kavanagh L K et al., 2016). Costimulatory signals boost the magnitude of the T-cell response and the generation of effector and memory T cells, and may promote cellular immune responses against tumors.

OX40 agonist antibody promotes T-cell activation and antitumor immunity in several clinical studies and synergizes with PD-L1 blockade to enhance the proliferation and function of exhausted CD8 T cells. The most common adverse effect of OX40 monotherapy are lymphopenia, fatigue, rash, and flu-like symptoms (grade 1-2) (Weinberg A D et al., 2000; Piconese S et al., 2008; Bulliard Y et al., 2014, Curti B D et al., 2013).

However, the combination of anti-OX40 with PD-1/PD-L1 checkpoint blockade produce a higher frequency of immune related adverse event (irAEs) than the respective individual treatments (Montler R et al., 2016).

Therefore, there is still a need for a therapy that increases the response to immunotherapeutic agents and reduces their side effects, adverse events and toxicity.

### SUMMARY OF THE INVENTION

In the present invention, the authors have identified that the combination of a specific caloric intake regime with at least one immunotherapeutic agent is effective in the treatment of cancer. Said specific caloric intake regime is based on a reduced daily caloric intake compared to a regular daily caloric intake, in particular it involves a specific daily caloric intake and a specific macronutrient intake as defined below.

The invention is based on the surprising finding that a reduced caloric intake or a fasting mimicking diet (FMD) enhances the therapeutic activity of immunotherapeutic agent in the treatment of cancer. Surprisingly and unexpectedly, the combination of the invention is effective on cancers characterized by resistance or partial response to the treatment with at least one immunotherapeutic agent.

Therefore the invention provides at least one reduced caloric intake cycle and at least one immunotherapeutic agent for use in the treatment of cancer wherein said at least one reduced caloric intake cycle comprises a first part with a regular caloric intake reduced by 30% to 70% and a second part with a regular caloric intake reduced by 40 to 97%.

Preferably said first part and/or said second part lasts for a period of 24 to 190 hours, preferably said first part and/or or said second part lasts for a period of 24 to 120 hours, preferably said first part and/or or said second part lasts for approximately 120 hours.

Preferably the at least one reduced caloric intake cycle is repeated from 1 to 30 times after respective periods of from 5 to 60 days.

In other words, each cycle may be separated by 5 to 60 days.

Preferably said at least immunotherapeutic agent is selected from the group consisting of: PD-inhibitor, PD-L1 inhibitor, CTLA-4 inhibitor, OX-40 activator.

Preferably the PD-1 inhibitor is selected from the group consisting of: nivolumab, pembrolizumab, cemiplimab, camrelizumab, sintilimab, toripalimab, tislelizumab, AK-105, dostarlimab, HLX-10, prolgolimab, SCTI-10A, spartalizumab, AK-103, AK-104, APL-501, balstilimab, BAT-1306, BI-754091, cetrelimab, CS-1003, GLS-010, MGA-012, pidilizumab, sasanlimab, AMG-404, BCD-217, BH-2950, budigalimab, CC-90006, F-520, HAB-21, HX-009, IBI-318, JTX-4014, LY-3434172, LZM-009, MEDI-5752, MGD-013, MGD-019, ONO-4685, RO-7121661, RO-7247669, sulituzumab, Sym-021, XmAb-20717, XmAb-23104 or a derivative thereof, or a combination thereof, the PD-L1 inhibitor is selected from the group consisting of: atezolizumab, durvalumab, avelumab, APL-502, bintrafusp alfa, CS-1001, KN-035, SHR-1316, BGBA-333, CX-072, GEN-1046, GS-4224, IO-103, IO-103+IO-120, KD-005, KLA-167, KN-046, lazertinib, STIA-1014, WP-1066, ADG-104, AK-106, BCD-135, CA-170, cosibelimab, FAZ-053, FPT-155, FS-118, HLX-20, IBI-318, INBRX-105, INCB-86550, JS-003, lodapolimab, LP-002, LY-3434172, MCLA-145, MSB-2311, RG-6084, SHR-1701, SL-279252, STIA-1015 or a derivative thereof, or a combination thereof, the CTLA-4 inhibitor is selected from the group consisting of: ipilimumab, tremelimumab, zalifrelimab, AK-104, BMS-986218, BMS-986249, KN-046, ADU-1604, AGEN-1181, ATOR-1015, BCD-145, BCD-217, FPT-155, HBM-4003, IBI-310, MEDI-5752, MGD-019, MK-1308, REGN-4659, RP-2, XmAb-20717, XmAb-22841, PSB-205, ALPN-202, APL-509, BPI-002, BT-001, CBT-103, CBT-107, CG-0161, HL-06, HLX-09, JS-007, KN-044, MV-049, ONC-392, PC-101, BJ-003, DB-002, IMT-400, JMW-3B3, TE-1254, AGEN-2041, FHTCT-4, HOR-010, PRS-010, SNCA-21 or a derivative thereof, or a combination thereof, the OX40 activator is selected from the group consisting of: BMS-986178, GSK-3174998, INCAGN-1949, KHK-4083, ABBV-368, ATOR-1015, DNX-2440, IBI-101, SL-279252, INBRX-106, AP-201, APVO-603, DPV-002, FS-120, HLX-51, JNJ-6892, MSB-013, OrthomAb, ABM-193, HuOHX-10, INV-531, SCB-340, ENUM-004, GBR-8383, KAHR-104, MEDI-6469, ZL-1101, efizonerimod alfa, tavolimab, vonlerolizumab or a derivative thereof, or a combination thereof.

Preferably the PD1 inhibitor is pembrolizumab, the PD-L1 inhibitor is atezolizumab, the CTLA-4 inhibitor is ipilimumab, the OX40 activator is BMS-986178.

Preferably the at least one reduced caloric intake cycle and at least one immunotherapeutic agent comprise administering a combination of:
PD-1 inhibitor and CTLA-4 inhibitor; or
PD-L1 inhibitor and CTLA-4 inhibitor; or
PD-L1 inhibitor and OX40 activator.

Preferably the at least one reduced caloric intake cycle and at least one immunotherapeutic agent for use as defined above, comprise administering a further therapeutic intervention.

Preferably said further therapeutic intervention is selected from the group consisting of: surgery, radiotherapy and at least one further therapeutic agent.

Preferably said further therapeutic agent is a further immune checkpoint inhibitor, an immune response stimulator, a targeted anticancer agent, a DNA Damage Response inhibitor and/or a chemotherapeutic agent.

Preferably said further immune checkpoint inhibitor is selected from the group consisting of: PD1 inhibitors, PDL1 inhibitors, CTLA-4 inhibitors, TIGIT inhibitors, ICOS inhibitors, TIM3 inhibitors, IDO1 inhibitors; said immune response stimulator is selected from the group consisting of: OX40 activators, GITR modulators, 4-1BB agonists; said targeted anticancer agent is selected from the group consisting of: PI3K inhibitors, HDAC inhibitors, EGFR inhibitors, BRAF inhibitors, MAPK inhibitors, CDK inhibitors, ER stress activators; said DNA Damage Response inhibitor is selected from the group consisting of: PARP inhibitors, CHK1 inhibitors, ATR inhibitors, Weel inhibitors; said chemotherapeutic agent is selected from the group consisting of: Alkylating agents, Antimetabolites, Anti-microtubule agents, Topoisomerase inhibitors, Cytotoxic antibiotics.

Preferably said cancer is characterized by resistance or partial response to the treatment with at least one immunotherapeutic agent, preferably said cancer is resistant or has a partial response to a least one PD-1 inhibitor, PD-L1 inhibitor, CTLA-4 inhibitor or OX-40 activator. Preferably said cancer is resistant or has a partial response to pembrolizumab and/or atezolizumab.

The cancer may be resistant to other agents such as commonly used chemotherapy.

Preferably said cancer is a solid or hematopoietic cancer, preferably the cancer is selected from the group consisting of: breast cancer, melanoma, lymphoma, lung cancer, non-small cell lung cancer (NSCLC), head and neck cancer, gastroesophageal cancer, bladder cancer and urothelial cancer, hepatocellular carcinoma and renal cell carcinoma.

The present invention provides a method of treatment of cancer comprising:
administering at least one reduced caloric intake cycle; and
administering at least one immunotherapeutic agent,

wherein said at least one reduced caloric intake cycle comprises a first part with a regular caloric intake reduced by 30% to 70% and a second part with a regular caloric intake reduced by 40 to 97%.

Preferably the cancer is characterized by resistance or partial response to the treatment with at least one immunotherapeutic agent.

Preferably said reduced caloric intake comprises a reduced protein intake and/or a reduced simple carbohydrate intake and/or an increased complex carbohydrate intake and/or an increased unsaturated fat intake.

Preferably said increased complex carbohydrate intake is approximately from 40% to 50% of total caloric intake, preferably said reduced protein intake is approximately from 9 to 11% of total caloric intake, preferably said increased complex carbohydrate intake is approximately from 43 to 47% of total caloric intake, preferably said increased unsaturated fat intake is approximately from 44 to 46% of total caloric intake.

In a preferred embodiment the reduced caloric intake is carried out by administering a specific regimen. Said regimen consists of a 4 days regimen. It provides approximately 1.100 kilocalories for a first day and less than 300 kilocalories per day for a second to fourth day of the diet (Table 1). It includes less than 30 grams of sugar on the first day; less than 5 grams of sugar on the second to fourth days; less than 30 grams of proteins on the first day; less than 5 grams of proteins on days the second to fourth days; less than 15 grams of saturated fats on the first day (Tables 2 and 3).

TABLE 1
Exemplary of Fasting mimicking diet (FMD) developed to induce
a fasting-like response while maximizing nourishment.

| | Day 1 | Day 2 | Day 3 | Day 4 |
|---|---|---|---|---|
| Total Calorie | 1.112 | 240 | 238 | 153 |

TABLE 2
The macronutrient content for each day of the 4 day FMD.

| | Day 1 | *Day 2, 3, 4 |
|---|---|---|
| Total Calorie | 1.112 | ~210 |
| Fats | ~61% | ~8% |
| Carbohydrates | ~31% | ~80% |
| (of which sugars) | (~9%) | ~6% |
| Proteins | ~9% | ~5% |

*Avarage values.

TABLE 3
The micronutrient content for each day of the 4 day
FMD regimen based on an average 180-200 lbs person.

| | Unit | Day 1 | Day 2, 3, 4* |
|---|---|---|---|
| Total Fat | (g) | 75 | 2 |
| % DV | | 96 | 2, 3 |
| Protein | (g) | 25 | 2, 6 |
| % DV | | 51 | 5 |
| Total Carbohydrate | (g) | 103 | 45, 6 |
| % DV | | 37 | 13, 3 |
| Sugars | (g) | 26, 30 | 3, 1 |
| Dietary Fiber | (g) | 32 | 5, 7 |
| % DV | | 113 | 19, 6 |
| VIT A | (IU) | 6.442 | 1.489 |
| % DV | | 716 | 157 |
| VIT C | (mg) | 9 | 15, 6 |
| % DV | | 101 | 14.35 |
| Calcium | (mg) | 445 | 52, 6 |
| % DV | | 34 | 5 |
| Iron | (mg) | 16 | 1, 6 |
| % DV | | 91 | 8 |
| Sodium | (mg) | 1.970 | 1.148 |
| % DV | | 82 | 49, 6 |
| Potassium | (mg) | 1.088 | 324, 3 |
| % DV | | 23 | 6, 6 |

% DV indicates the percent of daily value based on a 2.000 calorie diet updated to 2020 regulations.
*Avarage values

Embodiments and experiments illustrating the principles of the invention will be discussed with reference to the following figures.

### BRIEF DESCRIPTION OF THE FIGURES

FIG. 1. Fasting mimicking diet (FMD) and anti-PD-L1 synergize to mediate B16F10 melanoma tumour growth inhibition by increasing CD3+CD8+ Tumour infiltrating lymphocytes (TIL). Treatment schedule (A), mean tumour size (B), CD3+CD8+ TIL (C), Tregs (CD3+CD4+FoxP3+) (D) for C57BL/6 mice bearing B16F10 melanoma cells fed with standard diet (CTRL) or FMD and treated with anti-PD-L1 (100 μg/mouse), IgG (100 μg/mouse), anti-CD8 (200 μg/mouse). Data shown are pooled from 3 independent experiments with 4 mice per group. One-way ANOVA was used to evaluate statistical significance (*, P<0.05; **, P<0.01; ***, P<0.001; ****, P<0.0001; NS, P<0.1).

FIG. 2. Fasting mimicking diet in combination with Immune checkpoint blockade (ICB), anti-PD-L1/CTLA4 or anti-PD1/CTLA4, is effective in halting B16F10 melanoma tumour progression. Treatment schedule (A), mean tumour size (B), mean tumour weight (C) for C57BL/6 mice bearing B16F10 melanoma cells fed with standard diet (AL) or FMD and treated with immune checkpoint blockade (anti-PD-L1 100 μg/mouse, anti-PD1 100 μg/mouse and anti-CTLA-4 100 μg/mouse). Data shown are pooled from 3 independent experiments with 4 mice per group. One-way ANOVA was used to evaluate statistical significance (*, P<0.05; **, P<0.01; ***, P<0.001; ****, P<0.0001; NS, P<0.1).

FIG. 3. FMD in combination with anti-PD1/CTLA4 or anti-PD-L1/CTLA4 halts B16 tumour growth. Treatment schedule (A), mean tumour size (B), mean tumour weight (C) for C57BL/6 mice bearing B16F10 melanoma cells fed with standard diet (AL) or FMD and treated with immune checkpoint blockade (ICB) (anti-PD1 250 μg/mouse, and anti-CTLA-4 200 μg/mouse). Data shown are pooled from 3 independent experiments with 4 mice per group. One-way ANOVA was used to evaluate statistical significance (*, P<0.05; **, P<0.01; ***, P<0.001;****, P<0.0001; NS, P<0.1).

FIG. 4. One cycle of FMD enhances the antitumoral response of anti-PD1/CTLA4 treatment against B16 melanoma tumour. Treatment schedule (A), mean tumour size (B), mean tumour weight (C) for C57BL/6 mice bearing B16F10 melanoma cells fed with standard diet ad libitum (AL) or FMD and treated with immune checkpoint blockade ICB (anti-PD1 250 μg/mouse and anti-CTLA-4 200 μg/mouse).

FIG. 5. FMD boosts the anti-tumoral response of anti-PD-L1/OX40 therapy against 4T1 breast tumour and prevents spleen enlargement. Treatment schedule (A), mean tumour size (B), mean tumour weight (C) and mean spleen weight (D) for BALB/c mice bearing 4T1 breast tumour fed with standard diet ad libitum (AL) or FMD and treated with immune checkpoint blockade (anti-PD-L1 100 μg/mouse and anti-OX40 100 μg/mouse). Data shown are pooled from 3 independent experiments with 4 mice per group. One-way ANOVA was used to evaluate statistical significance (*, P<0.05; **, P<0.01; ***, P<0.001; ****, P<0.0001; NS, P<0.1).

FIG. 6. FMD increases CD8⁺ and CD8⁺CD25⁻CD44⁺ tumour infiltrating lymphocytes (TIL, activated by tumour antigens). CD3⁺ Tumour infiltrating lymphocytes (A), CD4⁺ TIL (B), CD8+ TIL (C) such cells include activated CD8⁺CD25⁻CD44⁺ cells, activated CD8+CD25−CD44+ TIL (D) for BALB/c mice bearing 4T1 breast tumour fed with standard diet ad libitum (AL) or FMD and treated with anti-PD-L1 (100 μg/mouse) and/or OX40 (100 μg/mouse). Data shown are pooled from 3 independent experiments with 4 mice per group. One-way ANOVA was used to evaluate statistical significance (*, P<0.05; **, P<0.01).

FIG. 7. FMD protects mice from anti-PDL1 and OX40 immune cytotoxicity. Beneficial effect of FMD in preventing anti-PD-L1 and/or anti-OX40 adverse effect on the survival of 4T1 allograft mice. Mice fed with standard diets ad libitum (AL) die upon the 4^{th} injection of anti-PD-L1 or anti-OX40, whereas mice fed with FMD (4 cycles) are much more tolerant and do not show any distress or adverse effect. Data shown are pooled from 3 independent experiments with 4 mice per group. One-way ANOVA was used to evaluate statistical significance (*, P<0.05; **, P<0.01; ***, P<0.001; ****, P<0.0001; NS, P<0.1).

### DETAILED DESCRIPTION OF THE INVENTION

The present invention provides a reduced caloric intake and at least one immunotherapeutic agent for the treatment of cancer.

Without being bound to any theory, the present data show that at least one reduced caloric intake cycle enhances the therapeutic activity of immunotherapeutic agent and/or increases CD8+ T cell population and/or reduces the toxicity of immunotherapeutic agent and/or prevents splenomegaly induced by immunotherapeutic agent.

The reduction is compared to a regular caloric intake per day. Regular caloric intake per day is between 1200 Kcal and 3000 Kcal. Preferably regular caloric intake per day (the range is based on age, sex and physical activity) is:

Age 4-8 years: 1200-2000 Kcal Age 9-13 years: 1800-2600 Kcal Age 19-30 years: 1800-3000 Kcal Age 31-50 years: 1800-2600 Kcal

+51 years: 1600-2600 Kcal.

In an embodiment, the reduced caloric intake for use according to the present invention lasts for a period of 24 to 190 hours, preferably said reduced caloric intake lasts for a period of 24 to 120 hours, preferably said reduced caloric intake lasts for approximately 120 hours.

In a preferred embodiment the period of reduced caloric intake is of 48 to 168 hours, preferably 120 hours.

Preferably the reduced caloric intake starts at least 24 hours before the immunotherapeutic agent is administered. Preferably the reduced caloric intake starts at least 48 hours before the immunotherapeutic agent is administered. Preferably the reduced caloric intake lasts at least 24 hours after the immunotherapeutic agent is administered, preferably it lasts at least 48, 72, 96, 120 hours after the immunotherapeutic agent is administered.

Preferably the reduced caloric intake is started one day before the immunotherapeutic agent is administered and continues for the following 2-4 days after immunotherapeutic agent administration (i.e. while the immunotherapeutic agent is most active). Preferably the reduced caloric intake consists of 4 days of low-calorie intake (50% of regular calorie intake on day 1, and 10% on days 2-4).

In the present invention, preferably, the reduced caloric intake is obtained by fasting or by means of dietetic food with reduced caloric and/or protein content but containing all necessary micronutrients to prevent malnutrition.

In the present invention, preferably, the reduced caloric intake is obtained by fasting mimicking diet (FMD). The term “fasting mimicking diet” (FMD) means a diet that mimics the effects of fasting typically by providing a subject with at most 50% of his normal caloric intake but with some nutritional component, so that fasting is mimicked while a subject is not completely starved. Examples of useful fasting mimicking and enhancing in the context of the present invention are set forth in U.S. patent application Ser. No. 14/273,946 filed May 9, 2014; Ser. No. 14/497,752 filed Sep. 26, 2014; Ser. No. 12/910,508 filed Oct. 22, 2010; Ser. No. 13/982,307 filed Feb. 8, 2012; Ser. No. 14/060,494 filed Oct. 22, 2013; Ser. No. 14/178,953 filed Feb. 12, 2014; Ser. No. 14/320,996 filed Jul. 1, 2014; Ser. No. 14/671,622 filed Mar. 27, 2015; the entire disclosure of these patent applications is hereby incorporated by reference. Additional examples of FMD diets are found in U.S. patent application Ser. No. 15/148,251 and WIPO Pub. No. WO 2011/050302 and WIPO Pub. No. WO 2011/050302; the entire disclosures of which are hereby incorporated by reference.

In the present invention the reduced caloric intake period is repeated one or more times after respective periods of 5-60 days, during which said mammal is given the agent while being subjected to a diet involving a regular caloric intake.

Preferably, the reduced caloric intake period is repeated from 1 to 3 times after respective period of 5-60 days.

Immunotherapy is the treatment of disease by activating or suppressing the immune system. Immunotherapies designed to elicit or amplify an immune response are classified as activation immunotherapies or immunostimulatory therapies, while immunotherapies that reduce or suppress are classified as suppression immunotherapies or immunosuppression therapies.

Preferably, immunotherapy relates to anticancer immunostimulatory therapies harnessing pre-existing, ineffective, immune responses by targeting the immune checkpoint pathways.

Preferably immunotherapeutic agents are: (1) drugs targeting the tumour immune evasion via blockade of negative regulatory signals (e.g., co-inhibitory checkpoints and tolerogenic enzymes) and (2) agents that directly stimulate immunogenic pathways (e.g., agonists of costimulatory receptors).

Additional immunostimulatory strategies include enhancers of antigen presentation (e.g., vaccines), the use of exogenous recombinant cytokines, oncolytic viruses, and cell therapies using native or modified antigen-competent immune cells.

The immunotherapeutic agents according to the invention can be antibodies or antibody derivatives or fragments thereof. Among the antibody fragments are functional equivalents or homologues of antibodies including any polypeptide comprising an immuno-globulin binding domain or peptides mimicking this binding domain. Chimeric molecules comprising an immunoglobulin binding domain, or equivalents, fused to another polypeptide are therefore included. Preferably, the antibody derivative comprises at least parts of the Fab fragment, preferably together with at least parts of the F(ab′)2 fragment and/or parts of the hinge region and/or the Fc part of a lambda or kappa antibody. Exemplary antibody molecules are intact immunoglobulin molecules and those portions of an immunoglobulin molecule that contains the para-tope, including those portions known as Fab, Fab′, F(ab′)2 and F (v). Preferably, the antibody is an IgG, IgM or IgA antibody.

The antibody or antibody derivative used according to the invention can also be a glycosylated antibody, wherein the glycosylation can also mimic an epitope of a carbohydrate epitope of a tumour associated antigen (TAA).

The antibody or antibody derivative can be of human or animal origin, preferably of mammalian origin, for example of mouse, rat, goat origin. It can be produced by hybridoma technology according to methods well known from the art or by recombinant expression using appropriate expression systems. Depending on the host system used, the antibody or antibody derivative can show specific glycosylation patterns.

The immunotherapeutic agent according to the invention can be an anti-idiotypic antibody, i.e. an ab2 and/or an idiotypic antibody having specificity for a tumour associated antigen, i.e. an ab1.

The immunotherapeutic agent according to the invention can also be a vaccine. This can be an antigenic structure, for example a TAA protein or polypeptide of a TAA which can either alone or together with a vaccine adjuvant induce an immune response against the antigen. The TAA antigen can be either isolated or recombinantly produced by known techniques.

The immunotherapeutic agent according to the invention can also be a small molecule.

In the context of the present invention, a “derivative” or “analogue” of an immunotherapeutic agent includes a chemical modification made for the purpose of improving its properties, especially its pharmacokinetic, pharmacodynamic, chemical or physical properties. For example, a derivative may be a chemical modification made to the inhibitor for the purpose of increasing its binding affinity towards the receptor, increase its bioavailability or half-life.

In an embodiment, the immunotherapeutic agent for use according to the present invention is selected from the group comprising: PD-1 inhibitor, PD-L1 inhibitor, CTLA-4 inhibitor, OX40 activator, or derivative thereof, or a combination thereof.

Programmed death-1/PD-1 (or CD279) is an immune checkpoint receptor and belongs to the B7-CD28 family of receptors. Upon binding to either of its two ligands, PD-L1 (known also as CD274 or B7-H1) and PD-L2 (known also as CD273, B7-DC or PDCD1LG2), a co-inhibitory signal is delivered. PD-1 is a 55-kDa monomeric type I surface transmembrane glycoprotein. PD-1 is an immune checkpoint and guards against autoimmunity through two mechanisms. First, it promotes apoptosis (programmed cell death) of antigen-specific T-cells in lymph nodes. Second, it reduces apoptosis in regulatory T cells (anti-inflammatory, suppressive T cells).

PD-1 inhibitors, a new class of drugs that block PD-1, activate the immune system to attack tumors and are used to treat certain types of cancer.

In an embodiment, the immunotherapeutic agent for use in the treatment of cancer is a PD-1 inhibitor.

In a preferred embodiment, the immunotherapeutic agent for use in the treatment of cancer is a PD-1 inhibitor selected from the group consisting of: nivolumab, pembrolizumab, cemiplimab, camrelizumab, sintilimab, toripalimab, tislelizumab, AK-105, dostarlimab, HLX-10, prolgolimab, SCTI-10A, spartalizumab, AK-103, AK-104, APL-501, balstilimab, BAT-1306, BI-754091, cetrelimab, CS-1003, GLS-010, MGA-012, pidilizumab, sasanlimab, AMG-404, BCD-217, BH-2950, budigalimab, CC-90006, F-520, HAB-21, HX-009, IBI-318, JTX-4014, LY-3434172, LZM-009, MEDI-5752, MGD-013, MGD-019, ONO-4685, RO-7121661, RO-7247669, sulituzumab, Sym-021, XmAb-20717, XmAb-23104 or a derivative thereof, or a combination thereof.

In a preferred embodiment, the immunotherapeutic agent for use in the treatment of cancer is pembrolizumab.

Programmed death-ligand 1 (PD-L1) also known as cluster of differentiation 274 (CD274) or B7 homolog 1 (B7-H1) is a protein that in humans is encoded by the CD274 gene.

The 40-kDa PD-L1 and the 25-kDa PD-L2 are both type I transmembrane proteins, containing extracellular IgV and IgC domains and a transmembrane domain. They lack an identifiable intracellular signalling domain. The two ligands share 37% identity with each other, but differ significantly in their affinity for PD-1 and their tissue specific expression.

PD-L1 expression/upregulation has been documented in various tumours, including melanoma, non-small cell lung cancer (NSCLC), breast cancer and squamous cell head and neck cancer. Binding of PD-L1 to its receptor suppresses T cell migration, proliferation, and secretion of cytotoxic mediators, and restricts tumour cell killing. Inhibitors of PD-1 and PD-L1 disrupt PD-1 axis thereby reverses T cell suppression and enhances endogenous antitumor immunity to unleash long-term antitumor responses for patients with a wide range of cancers.

In an embodiment, the immunotherapeutic agent for use in the treatment of cancer is a PD-1 inhibitor.

In a preferred embodiment, the immunotherapeutic agent for use in the treatment of cancer is a PD-L1 inhibitor selected from the group consisting of: atezolizumab, durvalumab, avelumab, APL-502, bintrafusp alfa, CS-1001, KN-035, SHR-1316, BGBA-333, CX-072, GEN-1046, GS-4224, 10-103, 10-103+IO-120, KD-005, KLA-167, KN-046, lazertinib, STIA-1014, WP-1066, ADG-104, AK-106, BCD-135, CA-170, cosibelimab, FAZ-053, FPT-155, FS-118, HLX-20, IBI-318, INBRX-105, INCB-86550, JS-003, lodapolimab, LP-002, LY-3434172, MCLA-145, MSB-2311, RG-6084, SHR-1701, SL-279252, STIA-1015 or a derivative thereof, or a combination thereof.

In a preferred embodiment, the immunotherapeutic agent for use in the treatment of cancer is atezolimumab.

CTLA4 or CTLA-4 (cytotoxic T-lymphocyte-associated protein 4), also known as CD152 (cluster of differentiation 152), 4 is a type I membrane protein that is expressed on activated T cells and monocytes, which mediates a local and temporary inhibition of the immune system.

CTLA4 is constitutively expressed in regulatory T cells but only upregulated in conventional T cells after activation—a phenomenon which is particularly notable in cancers. It acts as an “off” switch when bound to CD80 or CD86 on the surface of antigen-presenting cells.

CTLA-4 inhibitors can abolish the inhibition on the immune system, resulting in sustained immune-mediated antitumor activity.

In an embodiment, the immunotherapeutic agent for use in the treatment of cancer is a CTLA-4 inhibitor.

In a preferred embodiment, the immunotherapeutic agent for use in the treatment of cancer is a CTLA-4 inhibitor is selected from the group consisting of: ipilimumab, tremelimumab, zalifrelimab, AK-104, BMS-986218, BMS-986249, KN-046, ADU-1604, AGEN-1181, ATOR-1015, BCD-145, BCD-217, FPT-155, HBM-4003, IBI-310, MEDI-5752, MGD-019, MK-1308, REGN-4659, RP-2, XmAb-20717, XmAb-22841, PSB-205, ALPN-202, APL-509, BPI-002, BT-001, CBT-103, CBT-107, CG-0161, HL-06, HLX-09, JS-007, KN-044, MV-049, ONC-392, PC-101, BJ-003, DB-002, IMT-400, JMW-3B3, TE-1254, AGEN-2041, FHTCT-4, HOR-010, PRS-010, SNCA-21 or a derivative thereof, or a combination thereof.

In a preferred embodiment, the immunotherapeutic agent for use in the treatment of cancer is a CTLA-4 inhibitor is ipilimumab.

Tumour necrosis factor receptor superfamily, member 4 (TNFRSF4), also known as CD134 and OX40 receptor, is a member of the TNFR-superfamily of receptors which is not constitutively expressed on resting naïve T cells, unlike CD28. OX40 is a secondary costimulatory immune checkpoint molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels.

Agonistic antibodies to OX40 promote effector T-cell response and induce tumour regression. In an embodiment, the immunotherapeutic agent for use in the treatment of cancer is an OX40 activator.

In a preferred embodiment, the immunotherapeutic agent for use in the treatment of cancer is an OX40 activator selected from the group consisting of: BMS-986178, GSK-3174998, INCAGN-1949, KHK-4083, ABBV-368, ATOR-1015, DNX-2440, IBI-101, SL-279252, INBRX-106, AP-201, APVO-603, DPV-002, FS-120, HLX-51, JNJ-6892, MSB-013, OrthomAb, ABM-193, HuOHX-10, INV-531, SCB-340, ENUM-004, GBR-8383, KAHR-104, MEDI-6469, ZL-1101, efizonerimod alfa, tavolimab, vonlerolizumab or a derivative thereof, or a combination thereof.

In a preferred embodiment, the immunotherapeutic agent for use in the treatment of cancer is an OX40 activator is BMS-986178.

In one embodiment, the reduced caloric intake and at least one immunotherapeutic agent for use according to anyone of previous claims, comprises administering a combination of:
PD-1 inhibitor and CTLA-4 inhibitor; or
PD-L1 inhibitor and CTLA-4 inhibitor; or
PD-L1 inhibitor and OX40 activator.

In one embodiment, the reduced caloric intake and at least one immunotherapeutic agent for use according to the invention are combined with a further therapeutic intervention.

In a preferred embodiment, the reduced caloric intake and at least one immunotherapeutic agent for use according to the invention are combined with a further therapeutic intervention selected from the group consisting of: surgery, radiotherapy and at least one further therapeutic agent. Table 4 shows a list of drug classes that can be used in combination with the reduced caloric intake and the at least one immunotherapeutic agent for use according to the invention.

TABLE 4
Drug classes that can be used in combination with the reduced
caloric intake and the at least one immunotherapeutic agent.

| Drug Class | Drugs | Examples |
|---|---|---|
| Immune | PD1 inhibitors | anti-PD1 monoclonal antibodies |
| checkpoint | PDL1 inhibitors | anti-PDL1 monoclonal antibodies |
| inhibitors | CTLA-4 inhibitors | anti-CTLA4 monoclonal antibodies |
| | TIGIT inhibitors | anti-TIGIT monoclonal antibodies |
| | ICOS inhibitors | anti-ICOS monoclonal antibodies |
| | TIM3 inhibitors | anti-TIM3 monoclonal antibodies |
| | IDO1 inhibitors | Epacadostat monoclonal antibodies |
| Immune | OX40 activators | anti-OX40 monoclonal antibodies |
| response | GITR modulators | anti-GITR monoclonal antibodies |
| stimulators | 4-1BB agonists | anti-4-1BB monoclonal antibodies |
| Targeted | PI3K inhibitors | Buparlisib |
| anticancer | HDAC inhibitors | Entinostat |
| agent | EGFR inhibitors | Cetuximab |
| | BRAF inhibitors | Vemurafenib |
| | MAPK inhibitors | Dabrafenib |
| | CDK inhibitors | Palbociclib |
| | ER stress activators | Honokiol |
| DNA Damage | PARP inhibitors | Olaparib |
| Response | CHK1 inhibitors | LY2603618 |
| Inhibitors | ATR inhibitors | AZD6738 |
| | Wee1 inhibitors | AZD1775 |
| Chemo- | Alkylating agents | Cyclophosphamide |
| therapeutic | Antimetabolites | 5-fluorouracil |
| Drugs | Anti-microtubule | Paclitaxel |
| | agents | |
| | Topoisomerase | Camptothecin |
| | inhibitors | |
| | Cytotoxic | Bleomycin |
| | antibiotics | |

In a preferred embodiment, the reduced caloric intake and at least one immunotherapeutic agent for use according to the invention are combined with a further therapeutic agent is a further immune checkpoint inhibitor, an immune response stimulator, a targeted anticancer agent, a DNA Damage Response inhibitor and/or a chemotherapeutic agent.

In a preferred embodiment, the reduced caloric intake and at least one immunotherapeutic agent for use according to the invention are combined a further immune checkpoint inhibitor selected from the group consisting of: PD1 inhibitors, PDL1 inhibitors, CTLA-4 inhibitors, TIGIT inhibitors, ICOS inhibitors, TIM3 inhibitors, IDO1 inhibitors, and/or an immune response stimulator selected from the group consisting of: OX40 activators, GITR modulators, 4-1BB agonists, and/or a targeted anticancer agent selected from the group consisting of: PI3K inhibitors, HDAC inhibitors, EGFR inhibitors, BRAF inhibitors, MAPK inhibitors, CDK inhibitors, ER stress activators, and/or a DNA Damage Response inhibitor selected from the group consisting of: PARP inhibitors, CHK1 inhibitors, ATR inhibitors, Weel inhibitors, and/or a chemotherapeutic agent selected from the group consisting of: Alkylating agents, Antimetabolites, Anti-microtubule agents, Topoisomerase inhibitors, Cytotoxic antibiotics. Non limitative examples of immune checkpoint inhibitors include: nivolumab, pembrolizumab, cemiplimab, camrelizumab, sintilimab, toripalimab, tislelizumab, atezolizumab, durvalumab, avelumab, APL-502, bintrafusp alfa, ipilimumab, tremelimumab, zalifrelimab, AK-104, BMS-986207, tiragolumab, AB-154, AMG-570, ELN-21, ELN-12, darvadstrocel, indoximod, epacadostat.

Non limitative examples of immune response stimulators include: BMS-986178, GSK-3174998, INCAGN-1949, KHK-4083, ABBV-368, ATOR-1015, Utomilumab.

Non limitative examples of targeted anticancer agents include: alpelisib, copanlisib, rigosertib, dactolisib, duvelisib, entinostat, romidepsin, sodium phenylbutyrate, belinostat, panobinostat, osimertinib, cetuximab, panitumumab, erlotinib, vemurafenib, sorafenib, regorafenib, dabrafenib, miltefosine, brimapitide, acumapimod, palbociclib, abemaciclib, ribociclib, alvocidib, honokiol.

Non limitative examples of DNA Damage Response inhibitors include: olaparib, rucaparib camsylate, talazoparib, niraparib, LY2603618, AZD7762, SRA-737, CBP-501, ESP-01, prexasertib, AZD6738, berzosertib, M-4344, BAY-1895344, AZD1775, adavosertib, ZNC-3. Non limitative examples of chemotherapeutic agents include: Cisplatin, Carboplatin, Dicycloplatin, Oxaliplatin, Picoplatin, Satraplatin, Cyclophosphamide, Chlormethine, Uramustine, Melphalan, Chlorambucil, Ifosfamide, Bendamustine, Carmustine, Lomustine, Streptozocin, Busulfan, Procarbazine, Altretamine, Dacarbazine, Temozolomide, Mitozolomide.

Preferably, the reduced caloric intake and the at least one immunotherapeutic agent according to the present invention can be used in the treatment of a cancer characterized by resistance or partial response to the treatment with at least one immunotherapeutic agent.

Preferably, the reduced caloric intake and the at least one immunotherapeutic agent according to the present invention can be used in the treatment of a cancer characterized by a reduction in tumour mass or volume comprised between 0% and 90% after a treatment with at least one immunotherapeutic agent.

Preferably, the reduced caloric intake and the at least one immunotherapeutic agent according to the present invention can be used in the treatment of a cancer characterized by a reduction in tumour mass or volume comprised between 0% and 90% after a treatment with at least one immunotherapeutic agent selected from the group consisting of: PD-1 inhibitors, PD-L1 inhibitors, CTLA-4 inhibitors, OX40 activators.

Preferably, the reduced caloric intake and the at least one immunotherapeutic agent according to the present invention can be used in the treatment of a cancer selected from: breast cancer, melanoma, lymphoma, lung cancer, non-small cell lung cancer (NSCLC), head and neck cancer, gastroesophageal cancer, bladder cancer and urothelial cancer, hepatocellular carcinoma and renal cell carcinoma.

More preferably, the reduced caloric intake and the at least one immunotherapeutic agent according to the present invention can be used in the treatment of breast cancer or melanoma. The present invention further provides an in vitro method of treating a cancer cell with at least one agent as defined in above, comprising:
cultivating the cancer cell in a medium with reduced serum or glucose concentration; and
treating the cancer cell with the immunotherapeutic agent.
   wherein the serum concentration in the medium is less than 10% and the glucose concentration in less than 1 g/1 preferably the serum concentration is less than 5%, still preferably the serum concentration is 1% or less than 1%. Preferably the glucose concentration is less than 0.8 g/liter, preferably less than 0.6 g/liter, still preferably 0.5 g/liter, preferably less than 0.5 g/liter. Preferably the serum concentration in the medium is reduced by 10-90% or the glucose concentration in the medium is reduced by 20-90%, the reduction is in respect of normal or control concentrations (i.e. 10% of serum and 1 g/liter of glucose).

The present invention also provides a method of treatment of cancer comprising:
administering a reduced caloric intake; and
administering at least one immunotherapeutic agent.

wherein cancer is characterized by resistance or partial response to the treatment with at least one immunotherapeutic agent.

Wherein the immunotherapeutic agent is selected from the group consisting of: a PD-1 inhibitor, a PD-L1 inhibitor, a CTLA-4 inhibitor, an OX40 activator.

Wherein the reduced caloric intake lasts for a period of 24 hours to 190 hours and wherein said reduced caloric intake is a daily caloric intake reduced by 10 to 100%.

In the present invention a preferred reduced caloric intake is as follows: Day 1: 54% caloric intake, about 1,090 kcal (10% protein, 56% fat, 34% carbohydrate) Days 2-7: 20-34% caloric intake, about 426-725 kcal (5.3-9% protein, 26-44% fat, 27.6-47% carbohydrate).

The at least one immunotherapeutic agent for use according to the present invention can be incorporated into pharmaceutical compositions. Such compositions typically include the at least immunotherapeutic agent and at least one pharmaceutically acceptable carrier. As used herein the wording “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral, inhalation, transdermal (topical), transmucosal, and rectal administration.

In certain preferred embodiments, the pharmaceutical composition comprising at least one immunotherapeutic agent for use according to the present invention, used for parenteral, intradermal, or subcutaneous application, may further comprise one or more pharmaceutically acceptable carriers, exemplified by, but not limited to, lipid particles, lipid vesicles, liposomes, niosomes, sphingosomes, polymeric nanocarriers, nanoparticles, microparticles, nanocapsules, and nanospheres.

The pharmaceutical compositions of the present invention are preferably in the form of a single unit dosage form that contains an amount of the therapeutic agent that is effective to treat and/or prevent a cancer of the type described herein and at least one pharmaceutically acceptable excipient.

Suitable pharmaceutically acceptable excipients are those commonly known to the person skilled in the art for the preparation of compositions for parenteral, intradermal, subcutaneous, oral, transdermal, topical, transmucosal, and rectal administration.

By way of non-limiting example, said acceptable carriers can consists of binders, diluents, lubricants, glidants, disintegrants, solubilizing (wetting) agents, stabilizers, colorants, anti-caking agents, emulsifiers, thickeners and gelling agents, coating agents, humectants, sequestrants, and sweeteners.

The amount of the at least one immunotherapeutic agent in the pharmaceutical composition of the present invention will depend upon the nature and severity of the condition being treated, and on the nature of prior treatments which the patient has undergone. For a human patient, the attending physician will decide the dose of compound of the present invention with which to treat each individual patient. Initially, the attending physician can administer low doses and observe the patient's response. Larger doses may be administered until the optimal therapeutic effect is obtained for the patient, and at that point the dosage is not increased further. The duration of therapy using the pharmaceutical composition of the present invention will vary, depending on the severity of the disease being treated and the condition and potential idiosyncratic response of each individual patient.

In an embodiment, the invention provides a reduced caloric intake and at least one immunotherapeutic agent for use in the treatment of cancer, wherein the cancer is characterized by resistance or partial response to immunotherapy.

### Definitions

According to the present invention, preventing a disease refers to inhibiting completely, or in part, the development or progression of a disease, for example in a person who is known to have a predisposition to a disease. An example of a person with a known predisposition is someone with a history of cancer in the family, or who has been exposed to factors that predispose the subject to the development of a tumour.

Treating a disease refers to a therapeutic intervention that inhibits, or suppressed the growth of a tumour, eliminates a tumour, ameliorates at least one sign or symptom of a disease or pathological condition, or interferes with a pathophysiological process, after the disease or pathological condition has begun to develop.

Therapeutically effective dose is used in the context of the present invention to characterize an amount of the drug, which leads to complete or partial remission of the neoplastic disease. For instance, any statistically significant reduction in the mass or volume of the tumour or in the number of cancer cells indicates therapeutic efficacy in the context of the present invention.

Pharmaceutically acceptable is used in the context of the present invention to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

Resistance is used in the context of the present invention to refer to no variation in tumour mass or volume after a treatment with at least one immunotherapeutic agent.

Partial response is used in the context of the present invention to refer to a decrease in the size of a tumour, or in the extent of cancer in the body, in response to treatment with at least one immunotherapeutic agent. Preferably, partial response is used in the context of the present invention to refer to a reduction in tumour mass or volume comprised between 0% and 90% after a treatment with at least one immunotherapeutic agent. Partial response can also be called partial remission.

Immunotherapy is used in the context of the present invention to refer to the prevention or treatment of disease with agents (i.e. immunotherapeutic agents) that stimulate the immune response.

According to the present invention, simultaneous administration may, e.g., take place in the form of one fixed combination with two or more active ingredients, or by simultaneously administering two or more active ingredients that are formulated independently. Sequential use (administration) preferably means administration of one (or more) components of a combination at one time point, other components at a different time point, that is, in a chronically staggered manner, preferably such that the combination shows more efficiency than the single compounds administered independently (especially showing synergism). Separate use (administration) preferably means administration of the components of the combination independently of each other at different time points.

Also combinations of two or more of sequential, separate and simultaneous administration are possible, preferably such that the combination component-drugs show a joint therapeutic effect that exceeds the effect found when the combination component-drugs are used independently at time intervals so large that no mutual effect on their therapeutic efficiency can be found, a synergistic effect being especially preferred.

### Materials and Methods

### (1) Tumour Cell Lines.

The B16-F10 (ATCC® CRL6475™) and 4T1 (ATCC® CRL-2539™) tumour cell was obtained from ATCC. B16F10 are murine skin cancer cells used as model for human melanoma cancer. This cell line was derived from pulmonary melanoma nodule of the B16-FO parent cell line injected into C57BL/6 mouse tail vein. B16F10 cells are highly metastatic and will form tumors and metastases post implantation into syngenic C57BL/6 mice. 4T1 are murine breast cancer cells line derived from the mammary gland tissue of a mouse BALB/c and used as model for human triple negative breast cancer. 4T1 cells form tumors and spontaneous metastases post implantation into syngenic BALB/c mice.

B16 and 4T1 are also used as a pre-clinical model to study immunotherapy and both cells lines are resistant to immune checkpoint blockade therapy (anti-PD1, anti-PD-L1 and anti-CTLA4). Cells were grown in RPMI1640 or DMEM supplemented with 10% fetal bovine serum (FBS), 2 mM 1-glutamine, penicillin (100 U ml-1) and streptomycin (100 μg ml-1) at 37° C. with 5% CO2 and maintained at a confluence of 70-80%.

### (2) Tumour Implantation, Immune Checkpoint Blockade (ICB) Treatment and Tumour Volume Measurement.

C57BL/6J and BALB/c female mice, 6-8 weeks old, were purchased from Charles River and housed under pathogen-free conditions in Cogentech animal facility and with food and water ad libitum. All procedures were carried out in accordance with approved Institutional Animal Care and Use Committee (OPBA) animal protocols at IFOM—the FIRC Institute of Molecular Oncology.

In the vivo experiments, tumours were implanted in C57BL/6J mice by injecting subcutaneously (s.c.) 2×10⁵ B16 tumour cells per mouse into the right flank at day 0. When tumours were palpable, mice from the appropriate groups (5 mice per group) were treated intraperitoneally (i.p.) with anti-PD-1 antibody, anti-CTLA4 antibody and anti-PD-L1 antibody at different dose and timepoint according to designed experimental conditions.

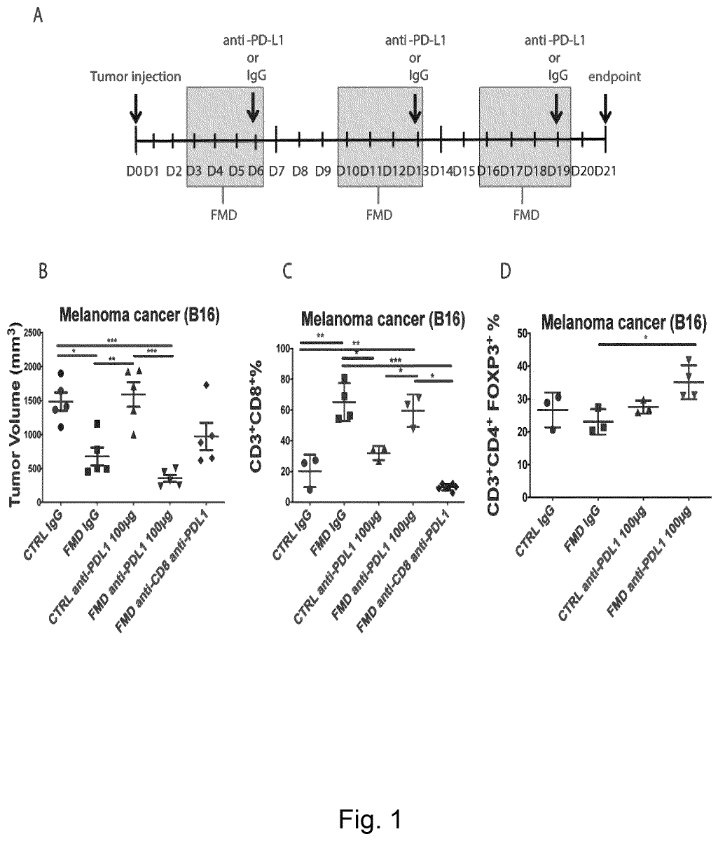

In the experiment shown in FIG. 1, IgG and anti-PD-L1 was administered 100 μg/mouse once a week for 3 weeks. The mice belonging to FMD group underwent 3 cycles of FMD, once per week.

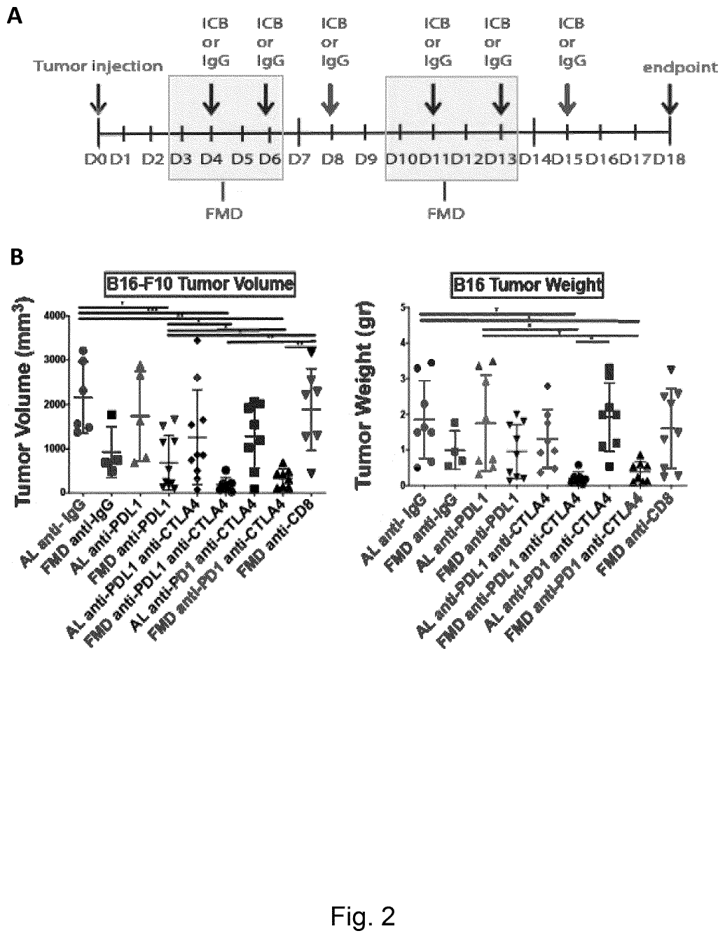

In the experiment of FIG. 2, IgG, anti-PD1, anti-PD-L1 and anti-CTLA4 were administered at a dose of 100 μg/mouse three times a week for 2 weeks. Anti-CD8 antibody was administered at a dose of 200 μg/mouse every 3 days. Mice belonging to FMD group underwent 2 cycles of FMD, once per week.

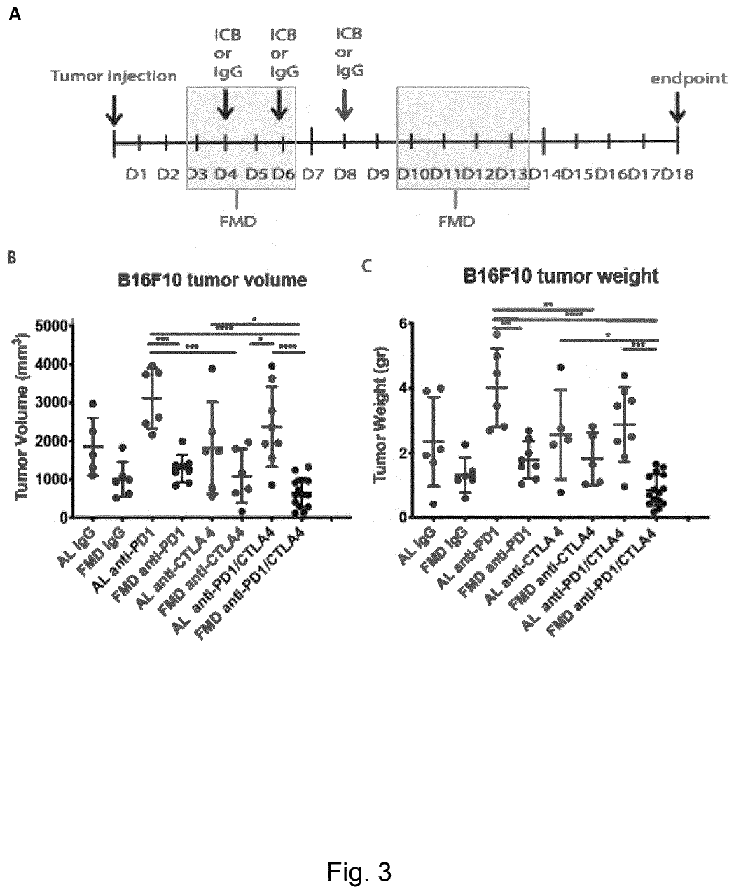

In the experiment of FIG. 3, mice were treated intraperitoneally (i.p.) with anti-PD-1 antibody (at the dose of 250 μg per mouse), anti-CTLA4 antibody (at dose of 200 μg per mouse the first injection and 100 μg per mouse the second and third injection) and IgG 450 μg per mouse.

Mice belonging to FMD group underwent 2 cycles of FMD, once per week.

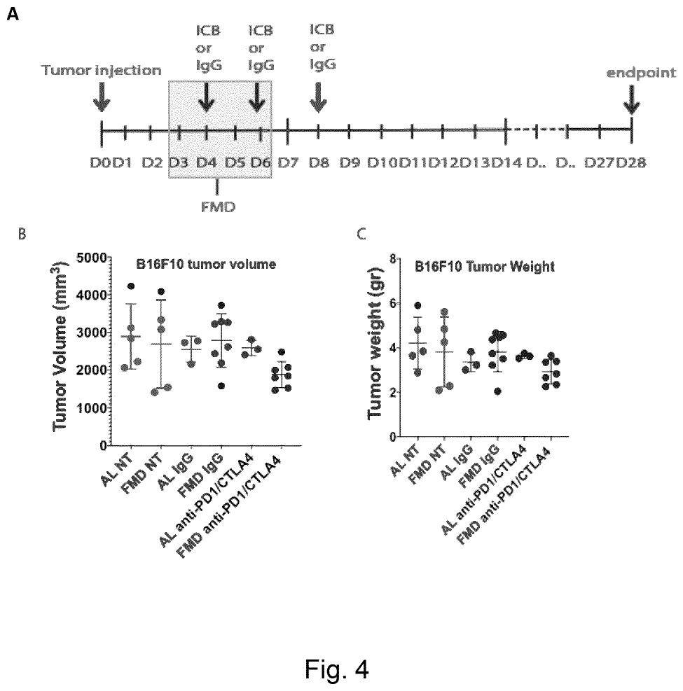

In the experiment of FIG. 4, mice were treated intraperitoneally (i.p.) with anti-PD-1 antibody (at the dose of 250 μg per mouse), anti-CTLA4 antibody (at dose of 200 μg per mouse the first injection and 100 μg per mouse the second and third injection) and IgG 450 μg per mouse.

Mice belonging to FMD group underwent 1 cycle of FMD.

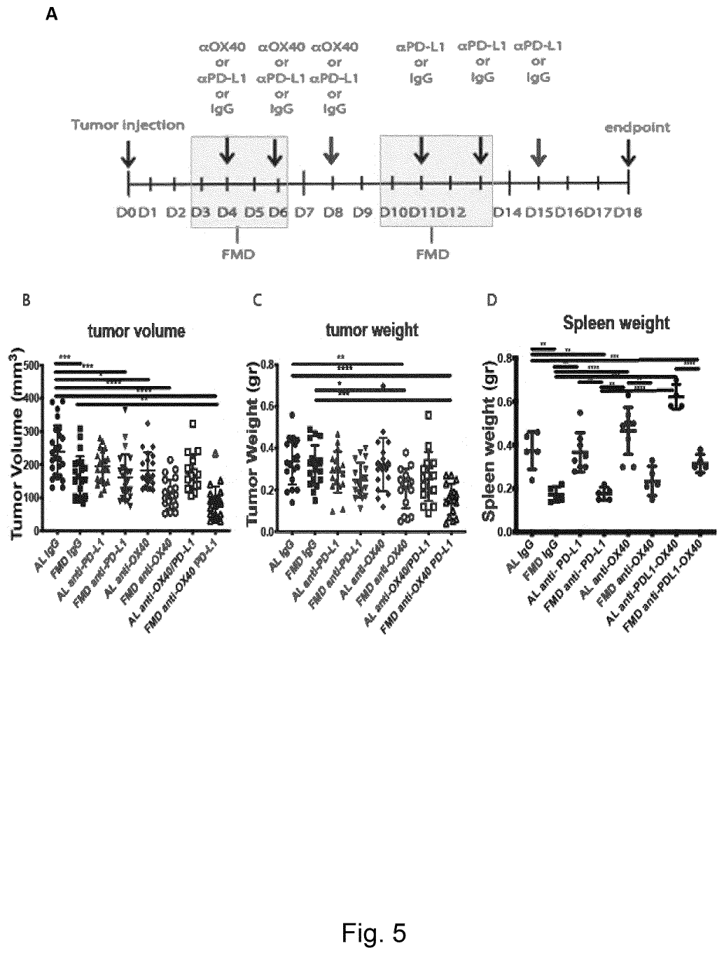

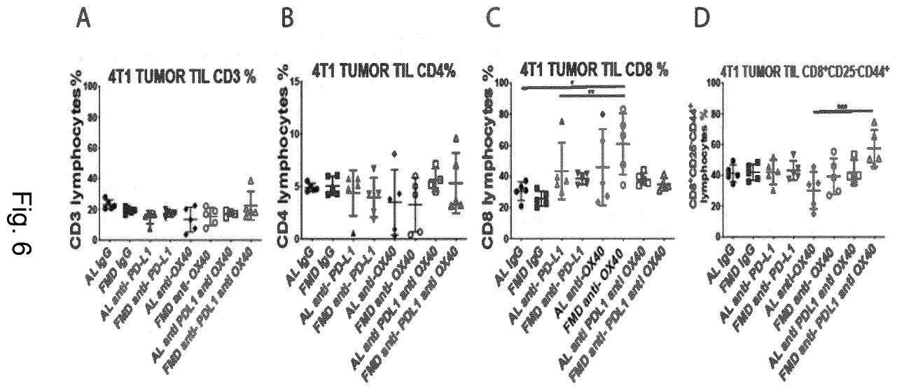

For triple negative breast cancer (TNBC) model, 3×10⁴ 4T1 cells were injected orthotopically into mouse mammary fat pad of 6-8-week-old BALB/c mice. When the tumours were palpable, mice were treated with IgG (100 μg per mouse) (anti-OX40 antibody 100 μg per mouse) and/or anti-PD-L1 antibody (100 μg per mouse) every other day for three times. Anti-OX40 was administered the first week while anti-PD-L1 was administered the second week to the group belonging to the combine treatment. Mice belonging to FMD group underwent 2 cycles of FMD, once per week (FIGS. 5 and 6).

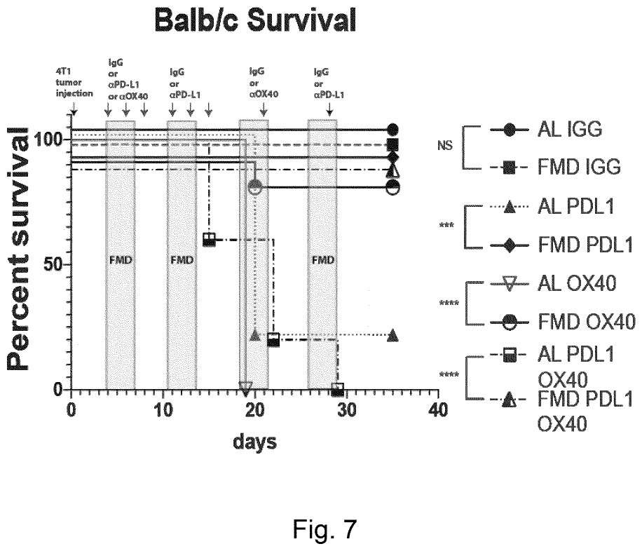

As reported in the experimental scheme of FIG. 7, 3 days after the injection of 4T1 cells into the mammary fat pad, the mice undergo 1 cycle of FMD (4 days) every week for 4 weeks and 3 treatments with IgG, anti-PD-L1 and/or anti-OX40 3 times a week on alternate days for 1 week. The mice belonging to anti-PD-L1/OX40 group are treated the first week 3 times with anti-OX40, the second week 3 times with anti-PD-L1, while the mice of the anti-PD-L1 and OX40 group do not receive any treatment the second week. Starting from the third week, the mice are treated with IgG, anti-PD-L1 or anti-OX40 1 time per week on the last day of FMD. Survival of mice over time was monitored.

Tumours were measured every 3-4 days using a digital caliper; tumour volume was calculated using the formula V=(L×W×H)/2, where V is tumour volume, L is the length of the tumour (longer diameter), W is the width of the tumour (shorter diameter) and H is the height (diameter of tumour perpendicular to length and width). Mice were monitored for tumour growth and survival. Mice were killed when tumour volume reached 1.5 cm³.

The tumor masses, collected at experimental endpoint, were weighed on an analytical balance in order to compare the volume and mass.

### (3) Flow Cytometry Analysis of Tumour-Infiltrating Lymphocytes and Apoptosis.

At experimental endpoint, the mice were sacrificed and all tumor masses removed. For the flow cytometry analysis of tumour-infiltrating lymphocytes, tumours were minced, B16 tumor masses, collected at experimental endpoint, were digested for 1 hour with Collagenase D (10 mg/ml) and DNAseI (10 μg/ml) whereas 4T1 tumours were processed with Miltenyi dissociation kit (130-096-730) according to manufacturer's instructions. 1−2×10⁶ cells per sample were stained with the LIVE/DEAD stain (Invitrogen L34959), CD45, CD3, CD8, CD4, CD44, FOXP3 and CD45 followed by fixation with formaldehyde.

Data acquisition was performed on Attune N×T Flow Cytometer. Results were analysed with the FlowJo software.

### (4) Immunotherapeutic Agents Used in the In Vivo Experiments.

In the above-described experimental procedures, the antibodies reported in Table 5 were used. The Catalogue number is from the antibody catalogue by BioXCell company, available at: https://bxcell.com/shop-products/

### (5) Standard Diet and FMD

TABLE 5
Agents used in the in vivo experiments.

| Name | Antigen | Clone | Catalogue No. |
|---|---|---|---|
| InVivoMAb antimouse PD-1 | PD-1 | J43 | BE0033-2 |
| (CD279) | (CD279) | | |
| InVivoMAb antimouse PD-L1 | PD-L1 | 10F.9G2 | BE0101 |
| (B7H1) | | | |
| InVivoMAb antimouse CTLA-4 | CTLA-4 | UC10- | BE0032 |
| (CD152) | (CD152) | 4F10-11 | |
| InVivoMAb anti-mouse OX-40 | OX-40 | OX-86 | BE0031 |
| (CD134) | (CD134) | | |
| InVivoPlus polyclonal antibody | N/A | N/A | BE0091 |
| Armenian Hamster IgG | | | |
| InVivoMAb anti-mouse CD8α | CD8 | 2.43 | BE0061 |

The mice were fed a standard diet (16% energy from proteins, 46% from carbohydrates, 38% from fat, total energy: 3.75 Kcal/g of food) or FMD throughout the course of the experiment. The FMD is a low-calorie diet based on vegetable compounds that is performed for 4 to 5 days. On the first day the mice are subjected to a calorie restriction diet that provides 50% of the daily energy requirement (5 g of “FMD day 1” diet as defined in Table 6, corresponding to 9.16 Kcal) determined according to the method described by Bachmanov, Behav Genet. 2002 November; 32(6): 435-443, while in the following three days the mice are fed a diet whose caloric intake is equal to 10% of the daily requirement (5 g of “FMD day 2-5” as defined in Table 6, corresponding to 1.80 Kcal). At the end of each FMD cycle mice are fed a standard diet for 3 days before starting a new FMD cycle. The number of FMD cycles vary according to the experimental conditions. In experiment of FIG. 1 there are 3 cycles of FMD, in experiment of figures 2, 3 and 5 animals were subjected to two FMD cycles, while in experiment of FIG. 4 it is only 1 FMD cycle was performed. In experiment of FIG. 7, 4 cycles of FMD were performed. Mice weight was monitored during all days of FMD and if the mice lost more than 20% of their body weight they were immediately fed with a standard diet.

TABLE 6
Energy density of FMD diets administered to mice.

| | |
|---|---|
| FMD day 1 | 1.83 Kcal/g of food (0.11 Kcal/g proteins; 0.53 Kcal/g |
| | carbohydrates; 1.19 Kcal/g fatty acids); |
| FMD day 2-5 | 0.36 Kcal/g of food (0.0023 Kcal/g proteins; 0.35 Kcal/g |
| | carbohydrates; 0.0023 Kcal/g fatty acids). |

### (6) Toxicity Assessment

In this experiment inventors evaluate the cytotoxicity of immunotherapy (anti-PDL1 and anti-OX40) in BALB/c mice carrying 4T1 tumors. As reported in the experimental scheme of FIG. 7, 3 days after the injection of 4T1 cells into the mammary fat pad, the mice undergo 1 cycle of FMD (4 days) every week for 4 weeks and 3 treatments with IgG, anti-PD-L1 and/or anti-OX40 3 times a week on alternate days for 1 week. The mice belonging to anti-PD-L1/OX40 group are treated the first week 3 times with anti-OX40, the second week 3 times with anti-PD-L1, while the mice of the anti-PD-L1 and OX40 group do not receive any treatment the second week. Starting from the third week, the mice are treated with IgG, anti-PD-L1 or anti-OX40 1 time per week on the last day of FMD. Survival of mice over time was monitored.

### EXAMPLES

### Example 1

The inventors investigated whether FMD could enhance the therapeutic activity of anti-PD-L1 in ICI (immune checkpoint inhibitor) or ICB (immune checkpoint blockade) resistant B16 melanoma. In the first set of experiments they injected subcutaneously B16F10 melanoma cells into C57B1/6 mice. When the tumour mass was palpable, mice underwent FMD cycles (reduced calorie intake by 50% day 1 and reduced calorie intake by 90% days 2, 3 and 4) every week and were treated 1 time a week for 3 weeks with anti-PD-L1 (100 μg/mouse). Anti-PD-L1 monotherapy has no effect on tumour growth in standard diet fed mice, whereas FMD not only delay tumour growth but increases the anti-tumour effect of anti PD-L1 (FIG. 1). In fact, FMD results in significantly reduced tumor progression alone in the FMD IgG control group compared to the standard diet control group (AL IgG). Treatment with anti-PD-L1 in combination with FMD further reduces the volume and progression of tumor masses compared to the FMD IgG control group, while it has no effect on tumor growth in the group fed the standard diet. Thus, FMD alone is able to block tumor growth and increase the anti-tumor efficacy of anti-PD-L1.

The effect of FMD in inhibiting tumor progression is mediated by the immune system as the percentage of CD8+ T lymphocytes increases in the tumor bed. CD8+ T lymphocytes constitute the lymphocyte population that mediates the anti-tumor response.

Indeed, CD8+ T cell lymphocytes depletion achieved by administrating a specific anti-CD8 antibody into mice, blunts the antitumor effect of FMD in combination with anti-PD-L1 (FIG. 1B, last lane) and the tumor masses in the group FMD anti-CD8 anti-PDL1 are larger than those in the FMD IgG group. This data demonstrated that the antitumor effect of FMD/anti-PD-L1 is immune mediated and CD8+ T cell dependent (FIG. 1 B, last lane).

### Example 2

The inventors next assess the effect of FMD and anti-PD-L1 treatment on tumour infiltrating lymphocytes which consist of a CD8+ and CD4+ T lymphocytes population that infiltrates the tumor bed and that regulates the anti-tumor immune response. Activated C D8+ T lymphocytes (CD8+CD44+) recognize and attack cancer cells by releasing cytolytic enzymes, whereas CD4+ T lymphocytes can differentiate into helper T lymphocytes and support the cytotoxic action of CD8 or can differentiate into Treg (CD4+FOXP3+) T lymphocytes which carry out an immunosuppressive activity and promote tumor growth. Monotherapy with anti-PD-L1 slightly increases the tumour infiltrating CD8+ cells compared to control-IgG treated mice (FIG. 1 C) but does not have any impact on CD4+ FoxP3+ infiltrating Tregs (FIG. 1D). Strikingly, FMD alone or in combination with anti-PD-L1 further increase CD8+ T cells population, compared to the single treatment.

### Example 3

The inventors evaluated whether FMD could potentiate the efficacy of anti-PDL1 and anti-PD1 in combination with anti-CTLA4. Mice were subcutaneously injected with 10⁵ B16F10 cells and subjected to 2 FMD cycles and treated with anti-PD1 or anti-PD-L1 (100 μg/mouse) and anti-CTLA4 every 2 days. FMD alone and in combination with anti PD1/CTLA4 and anti-PD-L1/CTLA4 was more effective in delaying tumour growth (volume and weight) compared to mice fed with standard diet and treated with anti-PD1/CTLA4 or anti-PD-L1/CTLA4. The inventors also confirm that the antitumor effect of FMD on tumour growth is immune mediated. Indeed the depletion of CD8+ T cell lymphocytes by administering specific anti-CD8 antibody into mice, reverts the anti-tumor effect of FMD on tumor growth (FIG. 2B, 2C).

FMD in combination with the anti-PD1/CTLA4 or anti-PD-L1/CTLA4 treatment improves the anti-tumor efficacy of the single anti-PD-L1 treatment. Anti-PD1/CTLA4 and anti-PD-L1/CTLA4 has minimal marginal effect on tumor growth in the standard diet fed mice group. The inventors also evaluate whether only 2 cycles of FMD in combination with higher dose of immunotherapics are able to recapitulate the same effects observed in FIG. 2.

In the experiment of FIG. 3, mice were treated intraperitoneally (i.p.) with anti-PD-1 antibody (at the dose of 250 μg per mouse), anti-CTLA4 antibody (at dose of 200 μg per mouse the first injection and 100 μg per mouse the second and third injection) and IgG 450 μg per mouse. The immunotherapeutics or IgG were administered only during week 1 (first cycle of FMD). Mice belonging to FMD group underwent 2 cycles of FMD, once per week.

Since the inventors administered the immunotherapeutics only during week 1, they increased the dose.

### Example 4

Inventors tested whether 1 cycle of FMD in combination with anti-PD1 (250 μg/mouse), anti-CTLA-4 (200 μg/mouse 1st treatment, 100 μg/mouse 2nd and 3rd treatment), administered every other day three times in 1 week, could have the same beneficial effect on tumor progression in B16F10 tumor-bearing mice.

The anti-PD1/CTLA-4 combined therapy exerted anti-tumour effect against B16F10 tumour in mice subjected to only 1 cycle of FMD (FIG. 4). Inventors did not observe any anti-tumour response to anti-PD1/CTLA-4 therapy in mice fed with standard diet. It is worth to note that 2 FMD cycles were able to decrease tumour size (volume and weight, FIG. 3), whereas 1 FMD cycle does not affect tumour growth compared to mice fed with standard diet (FIG. 4).

### Example 5

Then the inventors tested the efficacy of FMD in combination with anti-PDL1 and anti-OX40 against triple negative 4T1 breast cancer. BALB/c mice were injected with 5×10⁴ 4T1 cell in mammary fat pad and treated with anti-PD-L1 (100 μg/mouse) or anti-OX40 (100 μg/mouse) or both three times per week every other day for 1 week in the single therapy group and for 2 weeks in combined treatment. The combined anti-PDL1 and anti-OX40 treatment was administered not concurrently but sequentially: mice were treated with anti-OX40 1st week, whereas with anti-PD-L1 the 2nd week. The anti-PD-L1 and anti-OX40 monotherapy are ineffective at treating 4T1 breast cancer in BALB/c mice fed with standard diet. FMD in combination with anti-PD-L1 or OX40 reduces 4T1 tumour size whereas it elicits an additive effect in tumour volume and weight reduction when used in combination with anti-PD-L1 and anti-OX40 (FIGS. 5 B and C). FMD prevent splenomegaly specially in mice treated with the combination of anti-PD-L1 and OX40 (FIG. 5D). The spleen enlargement is generally due to an inflammatory state characterized by an increase in the red pulp, consisting mainly of red and white blood cells, and a strong reduction in the white pulp, an area of the spleen rich in B and T lymphocytes important for the adaptive immune response. In mice subjected to FMD the white pulp is more preserved and has multiple germinal centers, which indicate that splenic functionality is not compromised as in mice fed with a standard diet.

Analysis of tumor infiltrating lymphocytes (TILs) showed that the percentage of total CD3+ T lymphocytes (FIG. 6a) and of CD4⁺ T lymphocytes subpopulation (FIG. 6b) does not change in the various experimental conditions (standard diet or FMD; anti-PD-L1 and anti-OX40). On the other hand, FMD in combination with anti-PD-L1 increases the percentage of CD8⁺ cytotoxic T lymphocytes (FIG. 6c) while it does not affect CD8⁺CD44⁺ Cd25⁻T cells activation (FIG. 6d). In the anti-PD-L1 and anti-OX40 FMD group the population of infiltrating CD8⁺ T lymphocytes in the tumor bed does not change compared to the FMD/IgG controls (FIG. 6c), however a higher percentage of CD8+CD44+CD25⁻ active T lymphocytes is observed, which could explain the antitumoral effect observed on tumor masses growth in this group.

Analysis of tumour infiltrating lymphocytes showed that FMD plus anti-PD-L1 or anti-OX40 or both increase the percentage of CD8+ T cell in tumour bed, whereas FMD in combination with anti-PD-L1/OX40 promotes CD44+CD8+ T cell lymphocytes activation (FIG. 6).

Unexpectedly the inventors noticed that if they treat 4T1 tumor bearing mice with PD-L1 and anti OX40 in combination with FMD, on the 3rd and 4th week, 1 time per week, they observe that the anti-PD-L1 and anti-OX40 treatment on the 3^{rd} week result to be highly toxic for the animals fed with standard diet and cause suddenly death within 15′-30′ upon ICI injection. Furthermore, the anti-PDL1 therapy was lethal already the 2^{nd} week of treatment in some mice belonging to anti-PD-L1/OX40 combined treatment group (FIG. 7).

In other words, mice fed with standard diet are not very tolerant to the treatment with anti-PD-L1 and/or anti-OX40 already starting from the second week, while mice fed with FMD tolerate very well the treatment. Single anti-PD-L1 or anti-OX40 treatment is lethal to mice belonging to standard diet groups from the third week, while it has no detrimental effect in the groups fed with FMD.

As shown in FIG. 7, FMD (4 cycles separated by 3 days of food ad libitum) protects mice from anti-PD-L1 and OX40 induced-toxicity starting from the 3 week of treatment and boosts the antitumor efficacy of ICB therapy.

In patients, FMD regimen, may consist in a 5-day regimen, calorie-restricted (up to 1100 Kcal administered on day 1; up to 750 Kcal administered on days 2, 3, 4, 5), low-carbohydrate, low-protein diet.

In patients, FMD regimen, may also consist in a 4-days, calorie-restricted (up to 1100 Kcal administered on day 1; up to 750 Kcal administered on days 2, 3, 4), low-carbohydrate, low-protein diet.

For clinical trials on breast cancer or melanoma patients a FMD diet consisting of a 5-days, calorie-restricted (up to 600 Kcal administered on day 1; up to 300 Kcal administered on days 2, 3, 4, 5), low-carbohydrate, low-protein diet is adopted. FMD is repeated every 28 days for at least 2 times and immunotherapy is given on the third day of FMD. Immunotherapy is administered according to the doses reported in clinical protocol.

Breast cancer or melanoma patients can follow FMD diet consisting of a 5-days, calorie-restricted (up to 600 Kcal administered on day 1; up to 300 Kcal administered on days 2, 3, 4, 5), low-carbohydrate, low-protein diet, that can be repeated every 28 days for at least 2 times while immunotherapy is given on the third day of FMD. Immunotherapeutic agents are administered according to the doses known to the person with ordinary skill in the art.

### REFERENCES

1. Bianchi G, et al. Oncotarget. 2015 May 20; 6(14):11806-19.
2. Bulliard Y, et al., Immunol Cell Biol. 2014; 92(6):475-480.
3. Calabro L, et al., Lancet Oncol. 2013; 14:1104-11.
4. Curti B D, et al. Cancer Res. 2013; 73(24):7189-7198.
5. Di Biase S, et al. Cancer Cell. 2016 Jul. 11; 30(1):136-146.
6. Dougall W C, et al., Immunol Rev. 2017; 276(1):112-120.
7. El-Khoueiry A B et al. Lancet 389, 2492-2502 (2017).
8. Esensten J H, et al., Immunity. 2016; 44(5):973-988.
9. Foley E J Cancer Res 13, 835-837 (1953).
10. Fourcade J et al. J. Exp. Med 207, 2175-2186 (2010).
11. Garon E B et al. N. Engl. J. Med 372, 2018-2028 (2015).
12. Kim S T et al. Nat. Med 24, 1449-1458 (2018). [PubMed: 30013197]
13. Larkin J, et al., N Engl J Med. (2015) 373:1270-1. doi: 10.1056/NEJMc1509660
14. Lesokhin A M, et al., J. of Clinical Oncology, vol. 34, no. 23, pp. 2698-2704, 2016.
15. Lee C, et al. Science Translational Medicine. 2012; 4(124):124ra27.
16. Lee C, Oncogene. 2011; 30(30):3305-16. doi: 10.1038/onc.2011.91.
17. Levine M E, et al. Cell Metabolism. 2014; 19(3):407-17.
18. Longo V D, Science 2003; 299(5611):1342-6.
19. Matsuzaki J et al. Proc. Natl Acad. Sci. USA 107, 7875-7880 (2010).
20. Montler R, et al. Clin Transl Immunol. (2016) 5:e70. 10.1038/cti.2016.16
21. Motzer R J et al. J. Clin. Oncol 33, 1430-1437 (2015).
22. Mueller D L, Jenkins M K, Schwartz R H. Annu Rev Immunol. 1989; 7:445-480.
23. Piconese S, Valzasina B, Colombo M P. J Exp Med. 2008; 205(6):1505].
24. Postow M A, et al. N Engl J Med. (2015) 372:2006-17. doi: 10.1056/NEJMoa1414428
25. Raffaghello L, et al. PNAS 2008; 105(24):8215-20.
26. Ribas A, Hamid O, Daud A, Hodi F S, et al. JAMA. 2016 Apr. 19; 315(15):1600-9.
27. Ribas A & Wolchok J D Science 359, 1350-1355 (2018).
28. Robert C, et al. N Engl J Med. 2015 Jan. 22; 372(4):320-30.
29. Robert C, et al. N Engl J Med. 2015 Jun. 25; 372(26):2521-32.
30. Rosenberg J E et al. Lancet 387, 1909-1920 (2016).
31. Sade-Feldman M., et al., Nat Commun. 2017 Oct. 26; 8(1):1136.
32. Sangro B, et al. JHepatol. 2013; 59:81-8.
33. Sarnaik, A. A. et al. Clin. Cancer Res. 17, 896-906 (2011).
34. Schadendorf D, et al. J Clin Oncol. (2015) 33:1889-94.
35. Shim H S, Wei M, Brandhorst S, Longo V D. Cancer Res. 2015 Mar. 15; 75(6):1056-67
36. Slovin S F, et al. AnnOncol. 2013; 24:1813-21.
37. Smith H J Br. J. Cancer 20, 831-837 (1966).
38. Topalian S L et al. N. Engl. J. Med 366, 2443-2454 (2012).
39. Ward-Kavanagh L K, Lin W W, S ̆edy\acute{ } J R, Ware C F. Immunity. 2016; 44(5):1005-1019.
40. Walunas T L et al. Immunity 1, 405-413 (1994).
41. Weinberg A D, Rivera M M, Prell R, et al. J Immunol. 2000; 164(4):2160-2169.
42. Wikenheiser D J et al., Front Immunol. 2016; 7:304.
43. Wolchok J D, et al. N Engl J Med 2017; 377: 1345-1356.
44. Wolchok, J. D. et al. Lancet Oncol. 11, 155-164 (2010).
45. Yang J C, et al. JImmunother. 2007; 30:825-30.

1. A method for treating or preventing cancer in a patient in need of such prevention or treatment, comprising subjecting the patient to at least one reduced caloric intake cycle and at least one immunotherapeutic agent, wherein said at least one reduced caloric intake cycle comprises a first part with a regular caloric intake reduced by 30% to 70% and a second part with a regular caloric intake reduced by 40 to 97%.

2. The method of claim 1, wherein said first part and/or said second part lasts for a period of 24 to 190 hours.

3. The method of claim 1, wherein the at least one reduced caloric intake cycle is repeated from 1 to 30 times after respective periods of from 5 to 60 days.

4. The method of claim 1, wherein said at least immunotherapeutic agent is selected from the group consisting of: PD-1 inhibitor, PD-L1 inhibitor, CTLA-4 inhibitor, and OX-40 activator.

5. The method of claim 4, wherein the PD-1 inhibitor is selected from the group consisting of: nivolumab, pembrolizumab, cemiplimab, camrelizumab, sintilimab, toripalimab, tislelizumab, AK-105, dostarlimab, HLX-10, prolgolimab, SCTI-10A, spartalizumab, AK-103, AK-104, APL-501, balstilimab, BAT-1306, BI-754091, cetrelimab, CS-1003, GLS-010, MGA-012, pidilizumab, sasanlimab, AMG-404, BCD-217, BH-2950, budigalimab, CC-90006, F-520, HAB-21, HX-009, IBI-318, JTX-4014, LY-3434172, LZM-009, MEDI-5752, MGD-013, MGD-019, ONO-4685, RO-7121661, RO-7247669, sulituzumab, Sym-021, XmAb-20717, XmAb-23104 or a derivative thereof, or a combination thereof; the PD-L1 inhibitor is selected from the group consisting of: atezolizumab, durvalumab, avelumab, APL-502, bintrafusp alfa, CS-1001, KN-035, SHR-1316, BGBA-333, CX-072, GEN-1046, GS-4224, IO-103, IO-103+IO-120, KD-005, KLA-167, KN-046, lazertinib, STIA-1014, WP-1066, ADG-104, AK-106, BCD-135, CA-170, cosibelimab, FAZ-053, FPT-155, FS-118, HLX-20, IBI-318, INBRX-105, INCB-86550, JS-003, lodapolimab, LP-002, LY-3434172, MCLA-145, MSB-2311, RG-6084, SHR-1701, SL-279252, STIA-1015 or a derivative thereof, or a combination thereof; the CTLA-4 inhibitor is selected from the group consisting of: ipilimumab, tremelimumab, zalifrelimab, AK-104, BMS-986218, BMS-986249, KN-046, ADU-1604, AGEN-1181, ATOR-1015, BCD-145, BCD-217, FPT-155, HBM-4003, IBI-310, MEDI-5752, MGD-019, MK-1308, REGN-4659, RP-2, XmAb-20717, XmAb-22841, PSB-205, ALPN-202, APL-509, BPI-002, BT-001, CBT-103, CBT-107, CG-0161, HL-06, HLX-09, JS-007, KN-044, MV-049, ONC-392, PC-101, BJ-003, DB-002, IMT-400, JMW-3B3, TE-1254, AGEN-2041, FHTCT-4, HOR-010, PRS-010, SNCA-21 or a derivative thereof, or a combination thereof; the OX40 activator is selected from the group consisting of: BMS-986178, GSK-3174998, INCAGN-1949, KHK-4083, ABBV-368, ATOR-1015, DNX-2440, IBI-101, SL-279252, INBRX-106, AP-201, APVO-603, DPV-002, FS-120, HLX-51, JNJ-6892, MSB-013, OrthomAb, ABM-193, HuOHX-10, INV-531, SCB-340, ENUM-004, GBR-8383, KAHR-104, MEDI-6469, ZL-1101, efizonerimod alfa, tavolimab, vonlerolizumab or a derivative thereof, or a combination thereof.

6. The method according to claim 5, wherein the PD1 inhibitor is pembrolizumab, the PD-L1 inhibitor is atezolizumab, the CTLA-4 inhibitor is ipilimumab, and the OX40 activator is BMS-986178.

7. The method of claim 4, wherein the at least one immunotherapeutic agent is a combination of:
PD-1 inhibitor and CTLA-4 inhibitor; or
PD-L1 inhibitor and CTLA-4 inhibitor; or
PD-L1 inhibitor and OX40 activator.

8. The method of claim 1, further comprising administering a further therapeutic intervention and, said further therapeutic intervention is optionally selected from the group consisting of: surgery, radiotherapy and at least one further therapeutic agent, said further therapeutic agent is optionally a further immune checkpoint inhibitor, an immune response stimulator, a targeted anticancer agent, a DNA Damage Response inhibitor and/or a chemotherapeutic agent.

9. The method of claim 1, wherein said cancer is characterized by resistance or partial response to treatment with at least one immunotherapeutic agent.

10. The method of claim 1, wherein said cancer is a solid or hematopoietic cancer.

11. The method of claim 8, wherein said further therapeutic agent is a further immune checkpoint inhibitor and is selected from the group consisting of: PD1 inhibitors, PDL1 inhibitors, CTLA-4 inhibitors, TIGIT inhibitors, ICOS inhibitors, TIM3 inhibitors, and IDO1 inhibitors.

12. The method of claim 8, wherein said further therapeutic agent is an immune response stimulator and is selected from the group consisting of: OX40 activators, GITR modulators, and 4-1BB agonists.

13. The method of claim 8, wherein said further therapeutic agent is a targeted anticancer agent and is selected from the group consisting of: PI3K inhibitors, HDAC inhibitors, EGFR inhibitors, BRAF inhibitors, MAPK inhibitors, CDK inhibitors, and ER stress activators.

14. The method of claim 8, wherein said further therapeutic agent is a DNA Damage Response inhibitor and is selected from the group consisting of: PARP inhibitors, CHK1 inhibitors, ATR inhibitors, and Weel inhibitors.

15. The method of claim 8, wherein said further therapeutic agent is a chemotherapeutic agent and is selected from the group consisting of: Alkylating agents, Antimetabolites, Anti-microtubule agents, Topoisomerase inhibitors, and Cytotoxic antibiotics.

16. The method of claim 1, wherein said cancer is selected from the group consisting of: breast cancer, melanoma, lymphoma, lung cancer, non-small cell lung cancer (NSCLC), head and neck cancer, gastroesophageal cancer, bladder cancer and urothelial cancer, and hepatocellular carcinoma and renal cell carcinoma.
